(19) United States
(12) Patent Application Publication
(10) Pub. No.: US 2023/0221142 A1
(43) Pub. Date: Jul. 13, 2023

(54) STEP COUNTING JUDGMENT METHOD AND DEVICE AND COMPUTER-READABLE STORAGE MEDIUM

(71) Applicant: GOERTEK INC., Shandong (CN)

(72) Inventor: Yanhua TANG, Shandong (CN)

(73) Assignee: GOERTEK INC., Shandong (CN)

(21) Appl. No.: 17/997,210

(22) PCT Filed: Dec. 5, 2020
(86) PCT No.: PCT/CN2020/134137

§ 371 Date: Oct. 26, 2022

(30) Foreign Application Priority Data
Jun. 30, 2020 (CN) 202010619088.0

(51) Int. Cl. G01C 22/00; A61B 5/11

(52) U.S. Cl. CPC G01C 22/006; A61B 5/1123; A61B 2562/0219

(57) ABSTRACT

A step counting judgment method and device and a computer-readable storage medium are disclosed. The step counting judgment method comprises: acquiring an acceleration dataset corresponding to each axis of an acceleration sensor in a current step counting cycle; determining a characteristic value corresponding to each axis according to the acceleration dataset corresponding to each axis; comparing characteristic values of different axes, and determining that an axis corresponding to a maximum value in the characteristic values is a characteristic axis; judging validity of step counting data according to the characteristic axis and a reference axis; and performing a step counting operation when the step counting data is valid data. Aims to solve the problem of low step counting accuracy of step counting judgment devices, the present disclosure provides the step counting judgment method and device and the computer-readable storage medium.

### CROSS-REFERENCE TO RELATED APPLICATIONS

This Application is a U.S. National-Stage entry under 35 U.S.C. § 371 based on International Application No. PCT/CN2020/134137, filed Dec. 5, 2020 which was published under PCT Article 21(2) and which claims priority to Chinese Application No. 202010619088.0, filed Jun. 30, 2020, and which are all hereby incorporated herein in their entirety by reference.

### TECHNICAL FIELD

This application pertains to the technical field of wearable devices, and in particular, to a step counting judgment method and device and a computer-readable storage medium.

### BACKGROUND

With the development of electronic devices, users are paying more and more attention to their daily exercises. The step counting judgment device can record the number of walking or running steps in daily activities of users, so as to provide them with information of the number of moving steps, which is convenient for users to determine their exercise amount and energy consumption according to the information of the number of moving steps.

In the existing step counting methods, the acceleration sensor is typically used to collect the acceleration data of the user when walking, and the number of walking steps of the user is determined according to the acceleration data. However, in real life, when the user is performing actions such as housework or hand washing, the acceleration data collected by the acceleration sensor is similar to the acceleration data collected in walking, resulting in erroneous step counting. Moreover, since the acceleration data generated by some body movements is similar to the acceleration data generated by walking, the step counting judgment device cannot accurately record the number of walking steps of the user, resulting in the decrease in the accuracy of step counting.

The above content is only used to assist the understanding of the technical solutions of the present disclosure, and does not mean that the above content is acknowledged as the prior art. In addition, other objects, desirable features and characteristics will become apparent from the subsequent summary and detailed description, and the appended claims, taken in conjunction with the accompanying drawings and this background.

### SUMMARY

The present disclosure provides a step counting judgment method and device and a computer-readable storage medium, which aim to solve the problem in the prior art that step counting judgment devices have a low accuracy of step counting.

In order to achieve the above object, the present disclosure proposes a step counting judgment method, which comprises:
acquiring an acceleration dataset corresponding to each axis of an acceleration sensor in a current step counting cycle;
determining a characteristic value corresponding to each axis according to the acceleration dataset corresponding to each axis;
comparing characteristic values of different axes, and determining that an axis corresponding to a maximum value in the characteristic values is a characteristic axis;
judging validity of step counting data according to the characteristic axis and a reference axis; and
performing a step counting operation when the step counting data is valid data.

Optionally, the step of determining a characteristic value corresponding to each axis according to the acceleration dataset corresponding to each axis comprises:
determining a maximum value and a minimum value in the acceleration dataset according to the acceleration dataset corresponding to each axis; and
determining the characteristic value corresponding to the axis according to the maximum value and the minimum value in the acceleration dataset.

Optionally, the step of judging validity of step counting data according to the characteristic axis and a reference axis comprises:
determining whether the characteristic axis is the same as the reference axis, wherein when the characteristic axis is the same as the reference axis, it is determined that the step counting data is valid data.

Optionally, after the step of determining whether the characteristic axis is the same as the reference axis, the method further comprises:
when the characteristic axis is different from the reference axis, setting the characteristic axis as the reference axis of the next step counting cycle.

Optionally, the step of judging validity of step counting data according to the characteristic axis and a reference axis comprises:
determining whether the characteristic axis is the same as the reference axis;
when the characteristic axis is the same as the reference axis, making a number of buffering times unchanged and a number of repeating times increased by 1, wherein when the characteristic axis is the same as the reference axis, the method returns to execute the step of judging validity of step counting data according to the characteristic axis and a reference axis;
when the characteristic axis is different from the reference axis, making the number of buffering times increased by 1 and the number of repeating times increased by 1, wherein when the characteristic axis is different from the reference axis, the method returns to execute the step of judging validity of step counting data according to the characteristic axis and a reference axis; and
when the number of repeating times of the step counting cycle reaches a preset number of times, judging validity of the step counting data according to the number of buffering times and a buffering threshold, wherein when the number of buffering times is less than or equal to the buffering threshold, it is determined that the step counting data is valid data, and both the number of repeating times and the number of buffering times are reset to initial values.

Optionally, after the step of, when the number of repeating times of the step counting cycle reaches a preset number of times, judging validity of the step counting data according to the number of buffering times and a buffering threshold, the method further comprises:
when the number of buffering times is greater than the buffering threshold, resetting both the number of repeating times and the number of buffering times to initial values, and returning to execute the step of acquiring an acceleration dataset corresponding to each axis of an acceleration sensor in a current step counting cycle.

Optionally, the step of judging validity of step counting data according to the characteristic axis and a reference axis comprises:
determining whether the characteristic axis is the same as the reference axis;
when the characteristic axis is the same as the reference axis, adding a first characteristic value to a preset buffering array and making a number of repeating times increased by 1, wherein when the characteristic axis is the same as the reference axis, the method returns to execute the step of judging validity of step counting data according to the characteristic axis and a reference axis;
when the characteristic axis is different from the reference axis, adding a second characteristic value to the preset buffering array and making the number of repeating times increased by 1, wherein when the characteristic axis is different from the reference axis, the method returns to execute the step of judging validity of step counting data according to the characteristic axis and a reference axis, and the second characteristic value is not equal to the first characteristic value; and
when the number of repeating times of the step counting cycle reaches a preset number of times, judging validity of the step counting data according to a number of second characteristic values in the buffering array and a preset value, wherein when the number of the second characteristic values in the buffering array is less than the preset value, it is determined that the step counting data is valid data, the buffering array is cleared, and both the number of repeating times and the number of buffering times are reset to initial values.

Optionally, after the step of when the number of repeating times of the step counting cycle reaches a preset number of times, judging validity of the step counting data according to a number of second characteristic values in the buffering array and a preset value, the method further comprises:
when the number of the second characteristic values in the buffering array is greater than or equal to the preset value, clearing the buffering array, resetting the number of repeating times to an initial value, and returning to execute the step of acquiring an acceleration dataset corresponding to each axis of an acceleration sensor in a current step counting cycle.

In order to achieve the above object, the present disclosure proposes a step counting judgment device, which comprises: a memory, a processor and a step counting judgment program that is stored on the memory and can be executed by the processor, wherein when the processor executes the step counting judgment program, the step counting judgment method according to any one of the above embodiments is implemented.

In order to achieve the above object, the present disclosure proposes a computer-readable storage medium, wherein a step counting judgment program is stored on the computer-readable storage medium, and when the step counting judgment program is executed by a processor, the steps of the step counting judgment method according to any one of the above embodiments are implemented.

The present disclosure proposes a step counting judgment method, which comprises: acquiring an acceleration dataset corresponding to each axis of an acceleration sensor in a current step counting cycle; determining a characteristic value corresponding to each axis according to the acceleration dataset corresponding to each axis; comparing characteristic values of different axes, and determining that an axis corresponding to a maximum value in the characteristic values is a characteristic axis; judging validity of step counting data according to the characteristic axis and a reference axis; and performing a step counting operation when the step counting data is valid data. Before performing the step counting operation, by calculating the acquired acceleration dataset, and judging whether the user's motion state has changed based on the calculated characteristic axis. When the user's motion state has not changed, the subsequent step counting operation is executed; when the user's motion state has changed, it indicates that the user has changed from the walking state to another motion state, so there is no need to perform subsequent step counting operations. By judging the user's motion state based on the acceleration dataset, the erroneous step counting cases caused by the change of user's motion state is reduced, and thus the problem in the prior art that step counting judgment devices have a low accuracy of step counting can be solved.

### BRIEF DESCRIPTION OF DRAWINGS

The present invention will hereinafter be described in conjunction with the following drawing figures, wherein like numerals denote like elements, and:

FIG. 1 is a schematic view of a terminal structure of a hardware operating environment involved in a solution of an embodiment of the present disclosure;

FIG. 2 is a schematic flow chart of a step counting method according to a first embodiment of the present disclosure;

FIG. 3 is a schematic flow chart of a step counting method according to a second embodiment of the present disclosure;

FIG. 4 is a schematic flow chart of a step counting method according to a third embodiment of the present disclosure;

FIG. 5 is a schematic flow chart of a step counting method according to a fourth embodiment of the present disclosure;

FIG. 6 is a schematic flow chart of a step counting method according to a fifth embodiment of the present disclosure;

FIG. 7 is a schematic flow chart of a step counting method according to a sixth embodiment of the present disclosure;

FIG. 8 is a schematic flow chart of a step counting method according to a seventh embodiment of the present disclosure; and

FIG. 9 is a schematic flow chart of a step counting method according to an eighth embodiment of the present disclosure.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description.

The technical solutions in embodiments of the present disclosure will be described below with reference to the drawings in the embodiments of the present disclosure. Obviously, the embodiments as described below are merely part of, rather than all, embodiments of the present disclosure. Based on the embodiments of the present disclosure, any other embodiment obtained by a person of ordinary skill in the art without paying any creative effort shall fall within the protection scope of the present disclosure.

The technical solutions in embodiments of the present disclosure will be described clearly and completely below with reference to the drawings in the embodiments of the present disclosure. Obviously, the embodiments as described below are merely part of, rather than all, embodiments of the present disclosure. Based on the embodiments of the present disclosure, any other embodiment obtained by a person of ordinary skill in the art without paying any creative effort shall fall within the protection scope of the present disclosure.

It should be noted that all directional indications (such as up, down, left, right, front, back, etc.) in the embodiments of the present disclosure are only used to explain the relative positional relationship, the movement situation, etc. among various components under a certain posture (as shown in the accompanying drawings). If the specific posture changes, the directional indication will also change accordingly.

In addition, in the present disclosure, the terms “first” and “second” are used for descriptive purposes only and are not to be construed as indicating or implying relative importance or implicitly indicating the number of indicated technical features. Thus, a feature with “first” or “second” may explicitly or implicitly include at least one such a feature. In the description of the present disclosure, “a plurality of” means at least two, such as two, three, etc., unless expressly defined otherwise.

In the present disclosure, unless otherwise expressly specified and defined, the terms “connected”, “fixed” and the like should be understood in a broad sense, for example, it may be fixedly connected, or removably connected, or integrally connected; it may also be mechanically connected or electrically connected; it may also be directly connected or indirectly connected through an middleware; it may also be internally communicated or interacted between two components. For a person of ordinary skill in the art, the specific meaning of these terms in the present disclosure should be understood according to specific situations.

In addition, the technical solutions in various embodiments of the present disclosure can be combined with each other if their combination is realizable to a person of ordinary skill in the art. When the combination of technical solutions is contradictory or cannot be realized, it should be considered that such a combination of technical solutions does not exist and is not within the scope of protection claimed in the present disclosure.

It should be understood that the specific embodiments described herein are only used to explain the present disclosure, and is not used to limit the present disclosure.

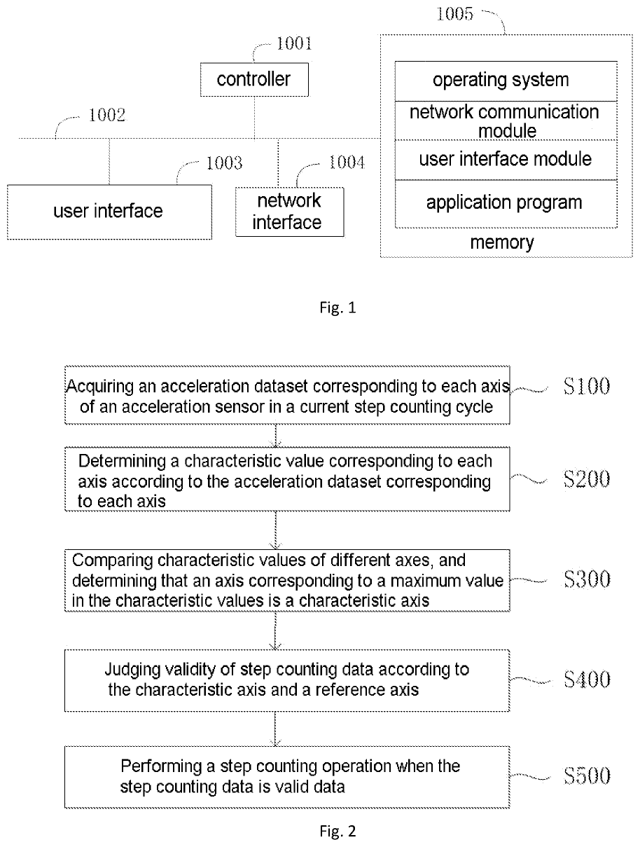

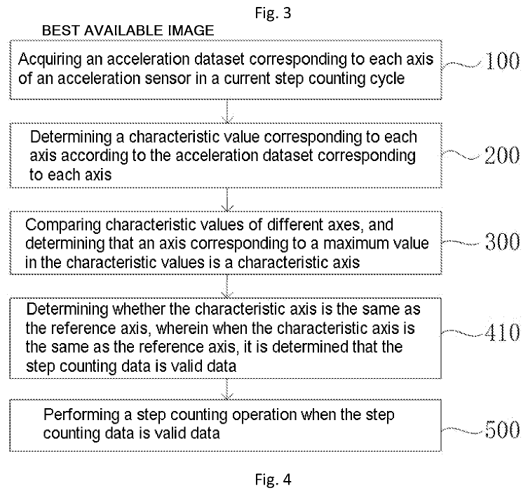

FIG. 1 is a schematic view of the device structure of a hardware operating environment involved in a solution of an embodiment of the present disclosure.

As shown in FIG. 1, the device may comprise: a controller 1001 (such as a CPU), a network interface 1004, a user interface 1003, a memory 1005, and a communication bus 1002. The communication bus 1002 is used to realize the connection and communication between these components. The user interface 1003 may comprise a display and an input unit (such as a keyboard). Optionally, the user interface 1003 may also comprise a standard wired interface and a wireless interface. Optionally, the network interface 1004 may comprise standard wired interface and wireless interface (such as Wi-Fi interface). The memory 1005 may be a high-speed RAM memory or a non-volatile memory, such as a disk memory. Optionally, the memory 1005 may be a storage device independent of the controller 1001.

A person of ordinary skill in the art may understand that the device structure shown in FIG. 1 does not constitute a limitation on the device, and it may include more or fewer components than those shown in the figure, or have different combinations or arrangements of components.

As shown in FIG. 1, the memory 1005, as a computer storage medium, may comprise: an operating system, a network communication module, a user interface module, and an application program.

In the server shown in FIG. 1, the network interface 1004 is mainly used to connect a background server and perform data communication with the background server. The user interface 1003 is mainly used to connect the client end (user end) and perform data communication with the client end. The controller 1001 may be used to invoke an application program stored in the memory 1005 and perform the following operations:
acquiring an acceleration dataset corresponding to each axis of an acceleration sensor in a current step counting cycle;
determining a characteristic value corresponding to each axis according to the acceleration dataset corresponding to each axis;
comparing characteristic values of different axes, and determining that an axis corresponding to a maximum value in the characteristic values is a characteristic axis;
judging validity of step counting data according to the characteristic axis and a reference axis; and
performing a step counting operation when the step counting data is valid data.

Further, the controller 1001 may invoke the application program stored in the memory 1005 and further perform the following operations:
determining a maximum value and a minimum value in the acceleration dataset according to the acceleration dataset corresponding to each axis; and
determining the characteristic value corresponding to the axis according to the maximum value and the minimum value in the acceleration dataset.

Further, the controller 1001 may invoke the application program stored in the memory 1005 and further perform the following operations:
determining whether the characteristic axis is the same as the reference axis, wherein when the characteristic axis is the same as the reference axis, it is determined that the step counting data is valid data.

Further, the controller 1001 may invoke the application program stored in the memory 1005 and further perform the following operations:
when the characteristic axis is different from the reference axis, setting the characteristic axis as the reference axis of the next step counting cycle.

Further, the controller 1001 may invoke the application program stored in the memory 1005 and further perform the following operations:
determining whether the characteristic axis is the same as the reference axis;
when the characteristic axis is the same as the reference axis, making a number of buffering times unchanged and a number of repeating times increased by 1, wherein when the characteristic axis is the same as the reference axis, the method returns to execute the step of judging validity of step counting data according to the characteristic axis and a reference axis;
when the characteristic axis is different from the reference axis, making the number of buffering times increased by 1 and the number of repeating times increased by 1, wherein when the characteristic axis is different from the reference axis, the method returns to execute the step of judging validity of step counting data according to the characteristic axis and a reference axis; and
when the number of repeating times of the step counting cycle reaches a preset number of times, judging validity of the step counting data according to the number of buffering times and a buffering threshold, wherein when the number of buffering times is less than or equal to the buffering threshold, it is determined that the step counting data is valid data, and both the number of repeating times and the number of buffering times are reset to initial values.

Further, the controller 1001 may invoke the application program stored in the memory 1005 and further perform the following operations:
when the number of buffering times is greater than the buffering threshold, resetting both the number of repeating times and the number of buffering times to initial values, and returning to execute the step of acquiring an acceleration dataset corresponding to each axis of an acceleration sensor in a current step counting cycle.

Further, the controller 1001 may invoke the application program stored in the memory 1005 and further perform the following operations:
determining whether the characteristic axis is the same as the reference axis;
when the characteristic axis is the same as the reference axis, adding a first characteristic value to a preset buffering array and making a number of repeating times increased by 1, wherein when the characteristic axis is the same as the reference axis, the method returns to execute the step of judging validity of step counting data according to the characteristic axis and a reference axis;
when the characteristic axis is different from the reference axis, adding a second characteristic value to the preset buffering array and making the number of repeating times increased by 1, wherein when the characteristic axis is different from the reference axis, the method returns to execute the step of judging validity of step counting data according to the characteristic axis and a reference axis, and the second characteristic value is not equal to the first characteristic value; and
when the number of repeating times of the step counting cycle reaches a preset number of times, judging validity of the step counting data according to a number of second characteristic values in the buffering array and a preset value, wherein when the number of the second characteristic values in the buffering array is less than the preset value, it is determined that the step counting data is valid data, the buffering array is cleared, and both the number of repeating times and the number of buffering times are reset to initial values.

Further, the controller 1001 may invoke the application program stored in the memory 1005 and further perform the following operations:
when the number of the second characteristic values in the buffering array is greater than or equal to the preset value, clearing the buffering array, resetting the number of repeating times to an initial value, and returning to execute the step of acquiring an acceleration dataset corresponding to each axis of an acceleration sensor in a current step counting cycle.

The present disclosure provides a step counting judgment method and device and a computer-readable storage medium.

### First Embodiment

Referring to FIG. 2, the step counting judgment method comprises:

S100, acquiring an acceleration dataset corresponding to each axis of an acceleration sensor in a current step counting cycle;

The step counting judgment device includes an acceleration sensor. Specifically, the acceleration sensor is used to measure the acceleration data of three axes, i.e., X-axis, Y-axis and Z-axis respectively. The acceleration data corresponding to the X-axis is a set of acceleration data in the X-axis direction varying with time measured by the acceleration sensor; the acceleration data corresponding to the Y-axis is a set of acceleration data in the Y-axis direction varying with time measured by the acceleration sensor; the acceleration data corresponding to the Z-axis is a set of acceleration data in the Z-axis direction varying with time measured by the acceleration sensor. After obtaining the acceleration datasets corresponding to the three axes, the acceleration sensor performs calculation on the three acceleration datasets to determine the total acceleration data.

S200, determining a characteristic value corresponding to each axis according to the acceleration dataset corresponding to each axis;

In order to facilitate the step counting judgment device to determine the characteristic value corresponding to each axis according to the acceleration data, it is necessary to calculate according to the acceleration data. Specifically, the acceleration data is the waveform data varying over a period of time. During the step counting operation, the effective wave peak is extracted from the acceleration data, and the step counting judgment data is determined according to the peak information and time information of the effective wave peak. In a specific embodiment, the characteristic axis is associated with the effective wave peak in the acceleration dataset. After the effective wave peak is determined through the acceleration dataset, the characteristic value is determined according to the effective wave peak.

S300, comparing characteristic values of different axes, and determining that an axis corresponding to a maximum value in the characteristic values is a characteristic axis;

When the user walks, the acceleration information of user detected by the acceleration sensor changes periodically. When the user is performing other actions, the limb activities for performing different actions are different, and thus the acceleration data detected by the acceleration sensor is also different. Specifically, the X-axis and Y-axis of the acceleration sensor are set as two axes perpendicular to each other in the horizontal direction, and the Z-axis is perpendicular to the plane formed by the X-axis and Y-axis. Preferably, the Z-axis direction is the vertical direction. When the user walks, the user detects that the acceleration changes greatly in the Z-axis direction, and does not change significantly in the X-axis and Y-axis. Therefore, it can be judged that when the user walks, the acceleration dataset of the acceleration sensor in the Z-axis direction is the largest.

S400, judging validity of step counting data according to the characteristic axis and a reference axis; and

S500, performing a step counting operation when the step counting data is valid data.

After determining the characteristic axis, it is necessary to judge the validity of the step counting data by comparing the characteristic axis with the reference axis. Specifically, when the characteristic axis is the same as the reference axis, it indicates that the step counting data collected by the acceleration sensor is valid data, and the data collected by the step counting is the valid data when the user is walking. Therefore, the step counting operation can be performed on the step counting data of the current step counting cycle.

The present disclosure proposes a step counting judgment method, which comprises: acquiring an acceleration dataset corresponding to each axis of an acceleration sensor in a current step counting cycle; determining a characteristic value corresponding to each axis according to the acceleration dataset corresponding to each axis; comparing characteristic values of different axes, and determining that an axis corresponding to a maximum value in the characteristic values is a characteristic axis; judging validity of step counting data according to the characteristic axis and a reference axis; and performing a step counting operation when the step counting data is valid data. Before performing the step counting operation, by calculating the acquired acceleration dataset, and judging whether the user's motion state has changed based on the calculated characteristic axis. When the user's motion state has not changed, the subsequent step counting operation is executed; when the user's motion state has changed, it indicates that the user has changed from the walking state to another motion state, so there is no need to perform subsequent step counting operations. By judging the user's motion state based on the acceleration dataset, the erroneous step counting cases caused by the change of user's motion state is reduced, and thus the problem in the prior art that step counting judgment devices have a low accuracy of step counting can be solved.

### Second Embodiment

In the first embodiment, the step S200 comprises:

S210, determining a maximum value and a minimum value in the acceleration dataset according to the acceleration dataset corresponding to each axis; and

S220, determining the characteristic value corresponding to the axis according to the maximum value and the minimum value in the acceleration dataset.

Specifically, the acceleration data stored in the acceleration dataset fluctuates up and down periodically after varying with time, thereby forming waveform information. In order to determine the walking condition of the user, the characteristic value corresponding to each axis can be determined according to the maximum and minimum values of the waveform information of each axis in the waveform information.

In a specific embodiment, the sampling frequency of the step counting judgment device is 25 Hz, which means that the acceleration sensor in the step counting judgment device collects acceleration data 25 times per second in different axes. If the step counting cycle is set to 1 second, then in one step counting cycle, the acceleration data collected 25 times constitutes a waveform, and the maximum value m and the minimum value n in the waveform are obtained respectively. The characteristic value is set to s=n+(m−n)/2, and the characteristic value corresponding to the axis is calculated based on the maximum value m and the minimum value n.

### Third Embodiment

Referring to FIG. 4, in the first embodiment, the step S400 comprises:

S410, determining whether the characteristic axis is the same as the reference axis, wherein when the characteristic axis is the same as the reference axis, it is determined that the step counting data is valid data.

When the characteristic axis is the same as the reference axis, it indicates that the user's motion state in the previous step counting cycle and the current step counting cycle has not changed. Therefore, it can be determined that the user is in a stable motion state in the previous step counting cycle and the current step counting cycle, so it is judged that the step counting data is valid data.

In a specific embodiment, when the user is walking, the movement range along the Z-axis direction is large, so the step counting judgment device sets the Z-axis as the reference axis. When the step counting judgment device determines that the Z-axis is the characteristic axis, the characteristic axis is the same as the reference axis, so it is judged that the current user is still walking, and thus the subsequent step counting function is executed to determine the number of walking steps of the user.

### Fourth Embodiment

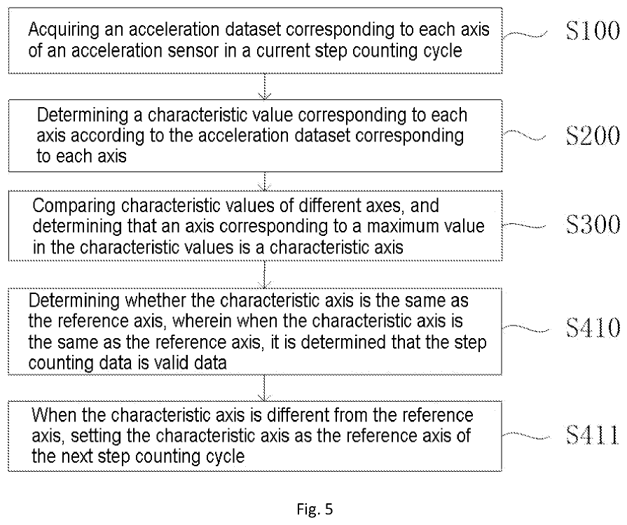

Referring to FIG. 5, in the third embodiment, after the step S410, the method further comprises:

S411, when the characteristic axis is different from the reference axis, setting the characteristic axis as the reference axis of the next step counting cycle.

After the step counting judgment device judges the characteristic axis of the current step counting cycle, when the characteristic axis is different from the reference axis, in order to judge the motion state of the next step counting cycle, it is necessary to set the characteristic axis of the current cycle as the reference axis of the next step counting cycle, so that it is convenient to judge whether the user's motion state of the next step counting cycle is the same as that of the current step counting cycle.

After the reference axis is set, the step of acquiring the acceleration dataset corresponding to each axis of the acceleration sensor in the current step counting cycle is repeatedly performed, so that each time after acquiring the acceleration dataset by the step counting judgment device, priority is given to the determination of the user's motion state, and continues to perform the step counting operation when the user's motion state is the same as the preset condition. Compared with directly performing the step counting operation, it can effectively avoid the erroneous step counting caused by the vibration that may be generated by the user in a non-walking state, thereby improving the accuracy of the step counting judgment device.

It can be understood that, besides that the reference axis can be determined through the previous step counting cycle, the corresponding axis can also be determined as the reference axis by presetting or defined by the user, so as to avoid the problem that when the user frequently switches the motion state, the step counting device changes the reference axis many times and thus the step counting judgment device makes misjudgment.

### Fifth Embodiment

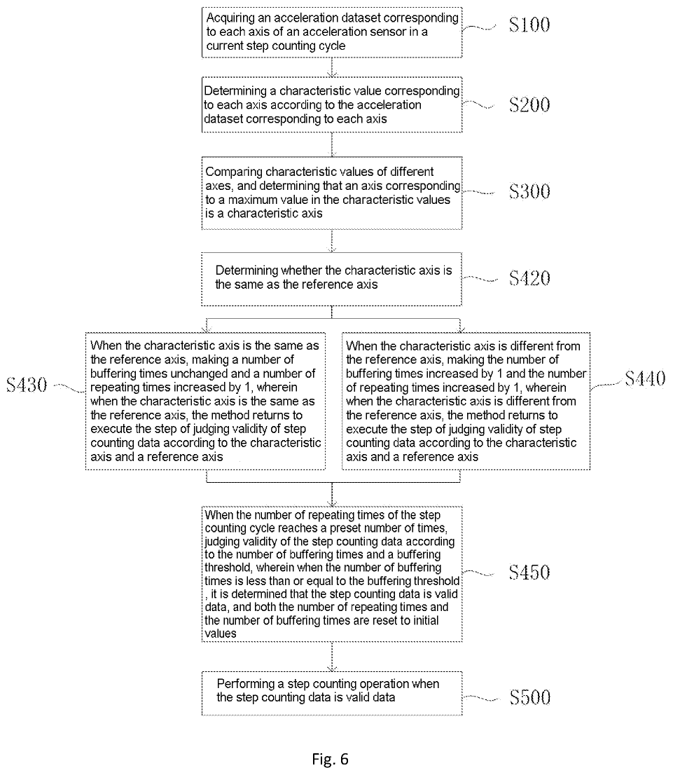

Referring to FIG. 6, in the first embodiment, the step S400 comprises:

S420, determining whether the characteristic axis is the same as the reference axis;

S430, when the characteristic axis is the same as the reference axis, making a number of buffering times unchanged and a number of repeating times increased by 1, wherein when the characteristic axis is the same as the reference axis, the method returns to execute the step of judging validity of step counting data according to the characteristic axis and a reference axis;

S440, when the characteristic axis is different from the reference axis, making the number of buffering times increased by 1 and the number of repeating times increased by 1, wherein when the characteristic axis is different from the reference axis, the method returns to execute the step of judging validity of step counting data according to the characteristic axis and a reference axis; and

S450, when the number of repeating times of the step counting cycle reaches a preset number of times, judging validity of the step counting data according to the number of buffering times and a buffering threshold, wherein when the number of buffering times is less than or equal to the buffering threshold, it is determined that the step counting data is valid data, and both the number of repeating times and the number of buffering times are reset to initial values.

During the step counting process, since the user's motion state will not change many times in a short time, a parameter, i.e., the number of buffering times, can be introduced to determine whether the user's motion state has changed based on the changes of the characteristic axis in multiple step counting cycles. Specifically, when judging the characteristic axis and the reference axis in one step counting cycle, it is set that when the characteristic axis is the same as the reference axis, the number of buffering times remains unchanged; when the characteristic axis is different from the reference axis, the number of buffering times is increased by 1. After the comparison between the characteristic axis and the reference axis is completed in each step counting cycle, the number of repeating times is increased by 1, and the method returns to step S100. After the number of repeating times of the step counting cycle reaches the preset number, the validity of the number of step counting cycles corresponding to the number of buffer times is judged according to the number of buffering times, and when the number of buffering times is less than or equal to the buffering threshold, it is determined that the step counting data of all the step counting cycles within the number of buffering times are valid data. Moreover, in order to facilitate the subsequent step counting operation, both the number of repeating times and the number of buffering times are reset to initial values.

In a specific embodiment, the preset number of times is set to 5, the step counting cycle is set to 1 second, the sampling frequency is set to 25 Hz, and the buffering threshold is set to 0, then the step counting judgment device acquires the acceleration data 25 times in each step counting cycle, and judges the characteristic axis once every second. When the characteristic axes of five step counting cycles are equal, the number of buffering times is 0, and then the step counting operation is started to perform on the step counting data of these 5 seconds.

### Sixth Embodiment

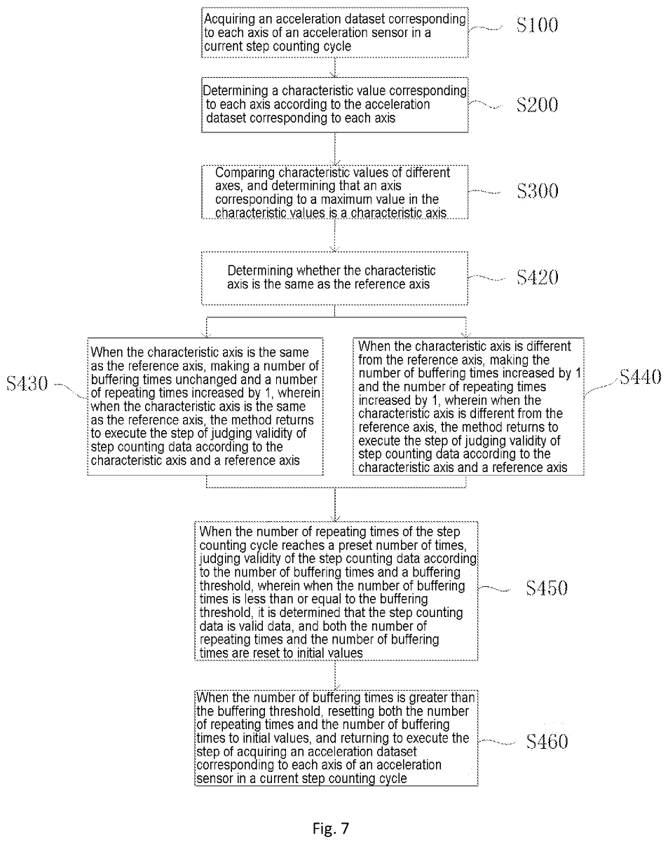

Referring to FIG. 7, in the fourth embodiment, after the step S450, the method further comprises:

S460, when the number of buffering times is greater than the buffering threshold, resetting both the number of repeating times and the number of buffering times to initial values, and returning to execute the step of acquiring an acceleration dataset corresponding to each axis of an acceleration sensor in a current step counting cycle.

Specifically, when the number of buffering times is greater than the buffering threshold, it indicates that the user's motion state has changed within the step counting cycles of the preset number of times. Therefore, the step counting operation is not performed within the step counting cycles of the preset number of times. In order to facilitate the operation of subsequently acquired data, both the number of repeating times of the step counting cycle and the number of buffering times are reset to initial values, so that it is convenient for the step counting judgment device to restart the judgment of the user's motion state.

In a specific embodiment, the preset number of times is set to 5 times, and the step counting cycle is set to 1 second. When the characteristic axes of five step counting cycles are not equal, the number of buffering times is greater than 0, it indicates that the user's motion state has changed within the 5 seconds, and the step counting operation will not be performed for the 5 seconds.

### Seventh Embodiment

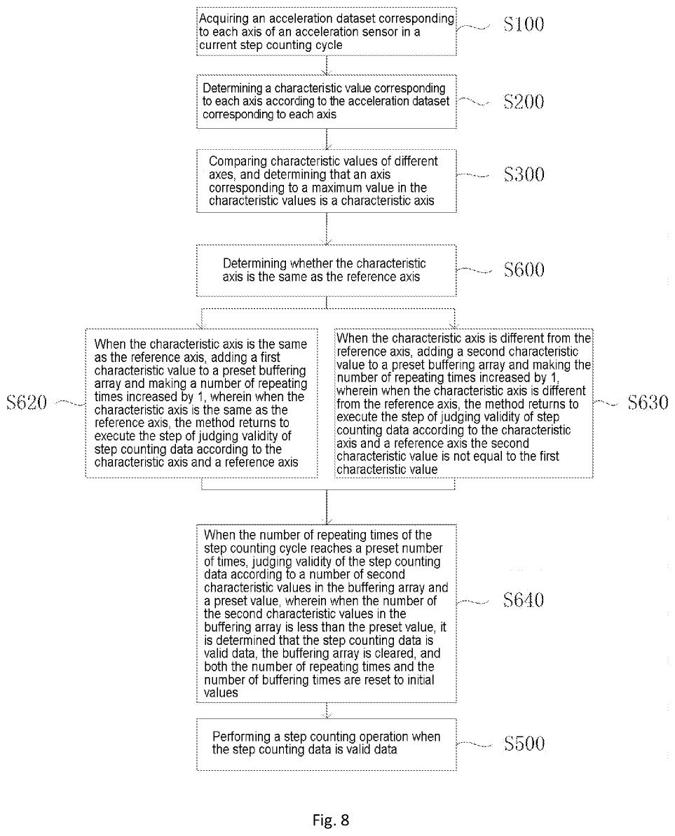

Referring to FIG. 8, in the first embodiment, the step S400 comprises:

S610, determining whether the characteristic axis is the same as the reference axis;

S620, when the characteristic axis is the same as the reference axis, adding a first characteristic value to a preset buffering array and making a number of repeating times increased by 1, wherein when the characteristic axis is the same as the reference axis, the method returns to execute the step of judging validity of step counting data according to the characteristic axis and a reference axis;

S630, when the characteristic axis is different from the reference axis, adding a second characteristic value to a preset buffering array and making the number of repeating times increased by 1, wherein when the characteristic axis is different from the reference axis, the method returns to execute the step of judging validity of step counting data according to the characteristic axis and a reference axis; the second characteristic value is not equal to the first characteristic value; and

S640, when the number of repeating times of the step counting cycle reaches a preset number of times, judging validity of the step counting data according to a number of second characteristic values in the buffering array and a preset value, wherein when the number of the second characteristic values in the buffering array is less than the preset value, it is determined that the step counting data is valid data, the buffering array is cleared, and both the number of repeating times and the number of buffering times are reset to initial values.

During the step counting process, since the user's motion state will not change many times in a short time, a parameter, i.e., the buffering array, can be introduced for determining of the user's motion state. When the judgment of the characteristic axis and the reference axis of each step counting cycle is completed, the first characteristic value or the second characteristic value is output according to whether the characteristic axis and the reference axis are the same. Specifically, when the characteristic axis is the same as the reference axis, the first characteristic value is output; when the characteristic axis is different from the reference axis, the second characteristic value is output. The second characteristic value is not equal to the first characteristic value. After step counting cycles of the preset number of times, the buffering array is judged. When the number of the second characteristic values in the buffering array is less than the preset value, it indicates that the user's motion state has not changed within step counting cycles of the preset number of times, and the step counting operation is continued to perform on the acceleration dataset. After the comparison between the characteristic axis and the reference axis is completed in each step counting cycle, the number of repeating times is increased by 1, and the method returns to step S100. After the number of repeating times of the step counting cycle reaches the preset number of times, the validity of the step counting data in the step counting cycles of number corresponding to the number of buffer times is judged according to the number of the second characteristic values in the buffering array, and when the number of buffering times is less than or equal to the buffering threshold, it is determined that the step counting data in all the step counting cycles of the number of buffering times are valid data. Moreover, in order to facilitate the subsequent step counting operation, the buffering array are cleared, and both the number of repeating times and the number of buffering times are reset to initial values.

In a specific embodiment, the preset number of times is set to 5, the step counting cycle is set to 1 second, the sampling frequency is set to 25 Hz, and the preset value is set to 1, then the step counting judgment device acquires the acceleration data 25 times in each step counting cycle, and judges the characteristic axis once every second. When the first characteristic value is set to 10, and the second characteristic value is set to 5, then after the judgment of the characteristic axis of five step counting cycles is completed, the buffering array is (10, 10, 10, 10, 10), which means that the user is still in the walking state within 5 seconds, and the step counting operation can be performed on the acceleration dataset within 5 seconds.

### Eighth Embodiment

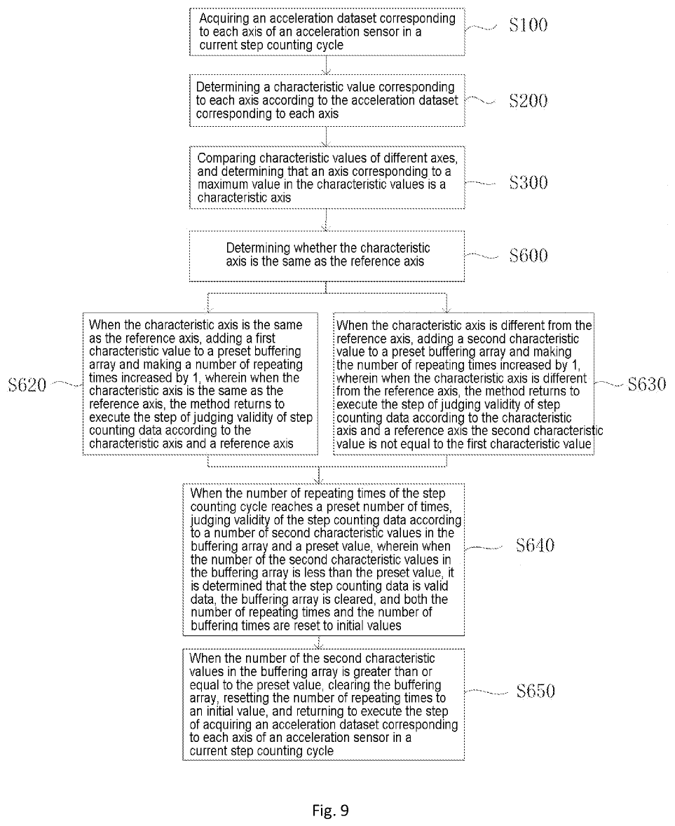

Referring to FIG. 9, in the seventh embodiment, after the step S640, the method further comprises:

S650, when the number of the second characteristic values in the buffering array is greater than or equal to the preset value, clearing the buffering array, resetting the number of repeating times to an initial value, and returning to execute the step of acquiring an acceleration dataset corresponding to each axis of an acceleration sensor in a current step counting cycle.

When the number of second characteristic values in the buffering array is greater than or equal to the preset value, it indicates that the user's motion state has changed within the preset number of step counting cycles, and it is necessary to stop the step counting operation on the acceleration dataset of the step counting cycle within the current preset number of times.

In a specific embodiment, the preset number of times is set to 5, the step counting cycle is set to 1 second, the sampling frequency is set to 25 Hz, and the buffering threshold is set to 0, then the step counting judgment device acquires the acceleration data 25 times in each step counting cycle, and judges the characteristic axis once every second. When the first characteristic value is set to 10, and the second characteristic value is set to 5, then after the judgment of the characteristic axis of five step counting cycles is completed, the buffering array is (10, 5, 10, 5, 10), it indicates that the user's motion state has changed for 2 seconds within 5 seconds, so the step counting operation is not performed on the acceleration dataset within the 5 seconds.

In order to achieve the above object, the present disclosure proposes a step counting judgment device, which comprises: a memory, a processor and a step counting judgment program that is stored on the memory and can be executed by the processor, wherein when the processor executes the step counting judgment program, the step counting judgment method according to any one of the above embodiments is implemented.

In order to achieve the above object, the present disclosure proposes a computer-readable storage medium, wherein a step counting judgment program is stored on the computer-readable storage medium, and when the step counting judgment program is executed by a processor, the steps of the step counting judgment method according to any one of the above embodiments are implemented.

In some optional embodiments, the processor may be a central processing unit (CPU), or other general-purpose processor, digital signal processor (DSP), application-specific integrated circuit (ASIC), field programmable gate array (FPGA), or other programmable logic device, discrete gate or transistor logic device, discrete hardware component, etc. A general purpose processor may be a microprocessor or any conventional processor or the like.

The memory may be an internal storage unit of the device, such as a hard disk or internal memory of the device. The memory may also be an external storage device of the device, such as a plug-in hard disk, a smart memory card (SMC), a secure digital (SD) card, a flash card, etc. equipped on the device. Further, the memory may include both an internal storage unit of the device and an external storage device. The memory is used to store the computer program and other programs and data required by the device.

The memory may also be used to temporarily store data that has been output or is to be output.

A person of ordinary skill in the art can clearly understand that, for the convenience and simplicity of description, only the division of the above-mentioned functional units or modules is illustrated as an example. In practical applications, the above-mentioned functions can be allocated to and completed by different functional units and modules, that is, the internal structure of the device is divided into different functional units or modules to complete all or part of the functions described above. In the embodiments, the functional units or modules may be integrated in one processing unit, or each unit may exist physically alone, or two or more units may be integrated into one unit. The above-mentioned integrated units may be realized in the form of hardware, or may be realized in the form of software functional units. In addition, the specific names of the functional units and modules are only for the convenience of distinguishing from each other, and are not used to limit the protection scope of the present disclosure. For the specific working process of the units and modules in the above-mentioned system, please refer to the corresponding process in the foregoing method embodiments, which will not be repeated here.

The above only describes the preferred embodiments of the present disclosure, and is not intended to limit the scope of the patent of the present disclosure. All technical solutions obtained by equivalent structural transformations made on the contents of the description and drawings of the present disclosure or direct/indirect application in other related technical fields under the inventive concept of the present disclosure shall fall within the scope of patent protection of the present application.

The steps of a method or algorithm described in conjunction with the embodiments disclosed herein may be directly implemented by hardware, by software module executed by a processor, or by a combination of hardware and software. The software module may be placed in a random access memory (RAM), an internal memory, read only memory (ROM), an electrically programmable ROM, an electrically erasable programmable ROM, a register, a hard disk, a removable disk, a CD-ROM, or any other form of storage medium known in the art.

It should also be noted that, relational terms such as first and second used herein are only to distinguish one entity or operation from another, and do not necessarily require or imply that there is such actual relationship or order among those entities or operations. Moreover, the terms “comprise”, “include” or any other variants are intended to cover non-exclusive inclusion, so that the process, method, article or apparatus including a series of elements may not only include those elements, but may also include other elements not stated explicitly, or elements inherent to the process, method, articles or apparatus. Without more limitations, an element defined by the phrase “comprising a . . . ” does not exclude the case that there are other same elements in the process, method, article or apparatus including the element.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the invention as set forth in the appended claims and their legal equivalents.

1. A step counting judgment method, comprising:
acquiring an acceleration dataset corresponding to each axis of an acceleration sensor in a current step counting cycle;
determining a characteristic value corresponding to each axis according to the acceleration dataset corresponding to each axis;
comparing characteristic values of different axes, and determining that an axis corresponding to a maximum value in the characteristic values is a characteristic axis;
judging validity of step counting data according to the characteristic axis and a reference axis; and
performing a step counting operation when the step counting data is valid data.

2. The step counting judgment method according to claim 1, wherein the step of determining a characteristic value corresponding to each axis according to the acceleration dataset corresponding to each axis comprises:
determining a maximum value and a minimum value in the acceleration dataset according to the acceleration dataset corresponding to each axis; and
determining the characteristic value corresponding to the axis according to the maximum value and the minimum value in the acceleration dataset.

3. The step counting judgment method according to claim 1, wherein after the step of determining whether the characteristic axis is the same as the reference axis, the method further comprises:
when the characteristic axis is different from the reference axis, setting the characteristic axis as the reference axis of the next step counting cycle.

4. The step counting judgment method according to claim 1, wherein the step of judging validity of step counting data according to the characteristic axis and the reference axis comprises:
determining whether the characteristic axis is the same as the reference axis;
when the characteristic axis is the same as the reference axis, making a number of buffering times unchanged and a number of repeating times increased by 1, wherein when the characteristic axis is the same as the reference axis, the method returns to execute the step of judging validity of step counting data according to the characteristic axis and a reference axis;
when the characteristic axis is different from the reference axis, making the number of buffering times increased by 1 and the number of repeating times increased by 1, wherein when the characteristic axis is different from the reference axis, the method returns to execute the step of judging validity of step counting data according to the characteristic axis and a reference axis; and
when the number of repeating times of the step counting cycle reaches a preset number of times, judging validity of the step counting data according to the number of buffering times and a buffering threshold, wherein when the number of buffering times is less than or equal to the buffering threshold, it is determined that the step counting data is valid data, and both the number of repeating times and the number of buffering times are reset to initial values.

5. The step counting judgment method according to claim 4, wherein after the step of, when the number of repeating times of the step counting cycle reaches a preset number of times, judging validity of the step counting data according to the number of buffering times and a buffering threshold, the method further comprises:
when the number of buffering times is greater than the buffering threshold, resetting both the number of repeating times and the number of buffering times to initial values, and returning to execute the step of acquiring an acceleration dataset corresponding to each axis of an acceleration sensor in a current step counting cycle.

6. The step counting judgment method according to claim 1, wherein the step of judging validity of step counting data according to the characteristic axis and a reference axis comprises:
determining whether the characteristic axis is the same as the reference axis;
when the characteristic axis is the same as the reference axis, adding a first characteristic value to a preset buffering array and making a number of repeating times increased by 1, wherein when the characteristic axis is the same as the reference axis, the method returns to execute the step of judging validity of step counting data according to the characteristic axis and a reference axis;
when the characteristic axis is different from the reference axis, adding a second characteristic value to the preset buffering array and making the number of repeating times increased by 1, wherein when the characteristic axis is different from the reference axis, the method returns to execute the step of judging validity of step counting data according to the characteristic axis and a reference axis, and the second characteristic value is not equal to the first characteristic value; and
when the number of repeating times of the step counting cycle reaches a preset number of times, judging validity of the step counting data according to a number of second characteristic values in the buffering array and a preset value, wherein when the number of the second characteristic values in the buffering array is less than the preset value, it is determined that the step counting data is valid data, the buffering array is cleared, and both the number of repeating times and the number of buffering times are reset to initial values.

7. The step counting judgment method according to claim 6, wherein after the step of when the number of repeating times of the step counting cycle reaches a preset number of times, judging validity of the step counting data according to a number of second characteristic values in the buffering array and a preset value, the method further comprises:
when the number of the second characteristic values in the buffering array is greater than or equal to the preset value, clearing the buffering array, resetting the number of repeating times to an initial value, and returning to execute the step of acquiring an acceleration dataset corresponding to each axis of an acceleration sensor in a current step counting cycle.

8. A step counting judgment device, comprising: a memory, a processor and a step counting judgment program that is stored on the memory and can be executed by the processor, wherein when the processor executes the step counting judgment program, the following steps are implemented:
acquiring an acceleration dataset corresponding to each axis of an acceleration sensor in a current step counting cycle;
determining a characteristic value corresponding to each axis according to the acceleration dataset corresponding to each axis;
comparing characteristic values of different axes, and determining that an axis corresponding to a maximum value in the characteristic values is a characteristic axis;
judging validity of step counting data according to the characteristic axis and a reference axis; and
performing a step counting operation when the step counting data is valid data.

9. A computer-readable storage medium, wherein a step counting judgment program is stored on the computer-readable storage medium, and when the step counting judgment program is executed by a processor, the following steps are implemented:
acquiring an acceleration dataset corresponding to each axis of an acceleration sensor in a current step counting cycle;
determining a characteristic value corresponding to each axis according to the acceleration dataset corresponding to each axis;
comparing characteristic values of different axes, and determining that an axis corresponding to a maximum value in the characteristic values is a characteristic axis;
judging validity of step counting data according to the characteristic axis and a reference axis; and
performing a step counting operation when the step counting data is valid data.

10. The step counting judgment device according to claim 8, wherein the step of determining a characteristic value corresponding to each axis according to the acceleration dataset corresponding to each axis comprises:
determining a maximum value and a minimum value in the acceleration dataset according to the acceleration dataset corresponding to each axis; and
determining the characteristic value corresponding to the axis according to the maximum value and the minimum value in the acceleration dataset.

11. The step counting judgment device according to claim 8, wherein after the step of determining whether the characteristic axis is the same as the reference axis, the method further comprises:
when the characteristic axis is different from the reference axis, setting the characteristic axis as the reference axis of the next step counting cycle.

12. The step counting judgment device according to claim 8, wherein the step of judging validity of step counting data according to the characteristic axis and the reference axis comprises:
determining whether the characteristic axis is the same as the reference axis;
when the characteristic axis is the same as the reference axis, making a number of buffering times unchanged and a number of repeating times increased by 1, wherein when the characteristic axis is the same as the reference axis, the method returns to execute the step of judging validity of step counting data according to the characteristic axis and a reference axis;
when the characteristic axis is different from the reference axis, making the number of buffering times increased by 1 and the number of repeating times increased by 1, wherein when the characteristic axis is different from the reference axis, the method returns to execute the step of judging validity of step counting data according to the characteristic axis and a reference axis; and
when the number of repeating times of the step counting cycle reaches a preset number of times, judging validity of the step counting data according to the number of buffering times and a buffering threshold, wherein when the number of buffering times is less than or equal to the buffering threshold, it is determined that the step counting data is valid data, and both the number of repeating times and the number of buffering times are reset to initial values.

13. The step counting judgment device according to claim 12, wherein after the step of, when the number of repeating times of the step counting cycle reaches a preset number of times, judging validity of the step counting data according to the number of buffering times and a buffering threshold, the method further comprises:
when the number of buffering times is greater than the buffering threshold, resetting both the number of repeating times and the number of buffering times to initial values, and returning to execute the step of acquiring an acceleration dataset corresponding to each axis of an acceleration sensor in a current step counting cycle.

14. The step counting judgment device according to claim 8, wherein the step of judging validity of step counting data according to the characteristic axis and a reference axis comprises:
determining whether the characteristic axis is the same as the reference axis;
when the characteristic axis is the same as the reference axis, adding a first characteristic value to a preset buffering array and making a number of repeating times increased by 1, wherein when the characteristic axis is the same as the reference axis, the method returns to execute the step of judging validity of step counting data according to the characteristic axis and a reference axis;
when the characteristic axis is different from the reference axis, adding a second characteristic value to the preset buffering array and making the number of repeating times increased by 1, wherein when the characteristic axis is different from the reference axis, the method returns to execute the step of judging validity of step counting data according to the characteristic axis and a reference axis, and the second characteristic value is not equal to the first characteristic value; and
when the number of repeating times of the step counting cycle reaches a preset number of times, judging validity of the step counting data according to a number of second characteristic values in the buffering array and a preset value, wherein when the number of the second characteristic values in the buffering array is less than the preset value, it is determined that the step counting data is valid data, the buffering array is cleared, and both the number of repeating times and the number of buffering times are reset to initial values.

15. The step counting judgment device according to claim 14, wherein after the step of when the number of repeating times of the step counting cycle reaches a preset number of times, judging validity of the step counting data according to a number of second characteristic values in the buffering array and a preset value, the method further comprises:
when the number of the second characteristic values in the buffering array is greater than or equal to the preset value, clearing the buffering array, resetting the number of repeating times to an initial value, and returning to execute the step of acquiring an acceleration dataset corresponding to each axis of an acceleration sensor in a current step counting cycle.

16. The computer-readable storage medium according to claim 9, wherein the step of determining a characteristic value corresponding to each axis according to the acceleration dataset corresponding to each axis comprises:
determining a maximum value and a minimum value in the acceleration dataset according to the acceleration dataset corresponding to each axis; and
determining the characteristic value corresponding to the axis according to the maximum value and the minimum value in the acceleration dataset.

17. The computer-readable storage medium according to claim 9, wherein after the step of determining whether the characteristic axis is the same as the reference axis, the method further comprises:
when the characteristic axis is different from the reference axis, setting the characteristic axis as the reference axis of the next step counting cycle.

18. The computer-readable storage medium according to claim 9, wherein the step of judging validity of step counting data according to the characteristic axis and the reference axis comprises:
determining whether the characteristic axis is the same as the reference axis;
when the characteristic axis is the same as the reference axis, making a number of buffering times unchanged and a number of repeating times increased by 1, wherein when the characteristic axis is the same as the reference axis, the method returns to execute the step of judging validity of step counting data according to the characteristic axis and a reference axis;
when the characteristic axis is different from the reference axis, making the number of buffering times increased by 1 and the number of repeating times increased by 1, wherein when the characteristic axis is different from the reference axis, the method returns to execute the step of judging validity of step counting data according to the characteristic axis and a reference axis; and
when the number of repeating times of the step counting cycle reaches a preset number of times, judging validity of the step counting data according to the number of buffering times and a buffering threshold, wherein when the number of buffering times is less than or equal to the buffering threshold, it is determined that the step counting data is valid data, and both the number of repeating times and the number of buffering times are reset to initial values.

19. The computer-readable storage medium according to claim 18, wherein after the step of, when the number of repeating times of the step counting cycle reaches a preset number of times, judging validity of the step counting data according to the number of buffering times and a buffering threshold, the method further comprises:
when the number of buffering times is greater than the buffering threshold, resetting both the number of repeating times and the number of buffering times to initial values, and returning to execute the step of acquiring an acceleration dataset corresponding to each axis of an acceleration sensor in a current step counting cycle.

20. The computer-readable storage medium according to claim 9, wherein the step of judging validity of step counting data according to the characteristic axis and a reference axis comprises:
determining whether the characteristic axis is the same as the reference axis;
when the characteristic axis is the same as the reference axis, adding a first characteristic value to a preset buffering array and making a number of repeating times increased by 1, wherein when the characteristic axis is the same as the reference axis, the method returns to execute the step of judging validity of step counting data according to the characteristic axis and a reference axis;
when the characteristic axis is different from the reference axis, adding a second characteristic value to the preset buffering array and making the number of repeating times increased by 1, wherein when the characteristic axis is different from the reference axis, the method returns to execute the step of judging validity of step counting data according to the characteristic axis and a reference axis, and the second characteristic value is not equal to the first characteristic value; and
when the number of repeating times of the step counting cycle reaches a preset number of times, judging validity of the step counting data according to a number of second characteristic values in the buffering array and a preset value, wherein when the number of the second characteristic values in the buffering array is less than the preset value, it is determined that the step counting data is valid data, the buffering array is cleared, and both the number of repeating times and the number of buffering times are reset to initial values.
